(19) United States
(12) Patent Application Publication
(10) Pub. No.: US 2023/0270569 A1
(43) Pub. Date: Aug. 31, 2023

(54) ORTHOPAEDIC JOINT DEVICE

(71) Applicant: Otto Bock Healthcare Products GmbH, Vienna (AT)

(72) Inventors: Juan-Pablo Mejia Nino, Moedling (AT); Dusan Vujanic, Vienna (AT)

(21) Appl. No.: 18/016,827

(22) PCT Filed: Jul. 20, 2021
(86) PCT No.: PCT/EP2021/070245

§ 371 Date: Jan. 18, 2023

(30) Foreign Application Priority Data
Jul. 20, 2020 (DE) 10 2020 004 337.2

(51) Int. Cl. A61F 2/60

(52) U.S. Cl. CPC A61F 2/604; A61F 2002/5006

(57) ABSTRACT

The invention relates to an orthopaedic joint device, comprising an upper part (2) and a lower part (3), which are mounted on one another so as to be pivotable about a joint pin (4) and between which a damper device (5) is situated in order to provide resistance against pivoting of the upper part (2) relative to the lower part (3), and the damper device (5) is mounted on the upper part (2) and the lower part (3) via fastening devices (6, 7), a fastening device (6) comprising a head (8) in which a bearing (9) is situated which is supported on a pin (10) that is mounted in the upper part (2) or lower part (3), characterised in that the pin (10) is mounted so as to be rotatable in the upper part (2) or lower part (3).

The invention relates to an orthopaedic joint device having an upper part and a lower part, which are mounted on one another so as to be pivotable about a joint axis and between which a damper device is arranged in order to provide a resistance to pivoting of the upper part relative to the lower part, and the damper device is mounted on the upper part and the lower part via fastening devices, wherein one fastening device has a head, in which a bearing is arranged, which is supported on an axle, which is mounted in the upper part or lower part.

Orthopaedic joint devices are used, in particular, for prostheses or orthoses, which also include exoskeletons, in order to allow two components to be pivoted with respect to one another. The joint device can be constructed in different ways; in addition to pure hinge joints, there are multiple-link joints, for example four-link joints, to enable even complex movements to be carried out. Multi-link joints are used, in particular, for simulating knee joints in order to enable the complex mechanics of a natural knee joint to be reproduced in a manner which is as true to nature as possible. Pure hinge joints or uniaxial joints with stationary joint axes are often used for ankle joints or elbow joints. Uniaxial knee joints with stationary axes are likewise frequently present, in particular in the case of prostheses or orthoses with sensor-controlled damper devices.

As a rule, the damper devices are designed as hydraulic or pneumatic dampers and have at least one cylinder, in which a piston is guided so as to be longitudinally displaceable. The damper device is mounted, on the one hand, on the lower part and, on the other hand, on the upper part of the orthopaedic joint device. It can be mounted either directly on the upper part or lower part of the orthopaedic joint device or on components or component parts of the orthopaedic joint device which are coupled to the upper part or lower part. Precisely in the case of multi-link systems, coupling of the damper device can also be accomplished via intermediate elements. As a rule, the damper device has a housing in which the cylinder is formed. A piston rod projects from the housing and is fastened to the upper part or the lower part by means of fastening devices. The housing can be designed to be pivotable on the lower part, and the piston rod has a head, as part of the fastening device, which is mounted so as to be pivotable about an axle which is supported on the upper part or the lower part. The rotatability of the axle is necessary in order to be able to convert the rotary movement about the joint axis into a linear movement for retracting and extending the piston rod.

A recurring problem in the case of hydraulic damper devices in orthopaedic joint devices is the occurrence of transverse forces and torsional moments due to the pivoting of the upper part and lower part about the joint axis and due to manufacturing inaccuracies and deformations during loading. Thus, for example, lower parts can be designed as a frame made of a fiber-reinforced plastic, thereby making it possible to provide particularly light-weight joint devices. Problems here can be caused by a lack of rigidity or comparatively high flexibility, which can lead to torsional moments and transverse forces in the region of the damper device during the pivoting of the upper part relative to the lower part.

The arrangement of a bearing in a head at the end of a piston rod, which is designed as a ball head bearing, is known from the prior art. This makes it possible, in particular, to compensate torsional moments around the longitudinal extent of the piston rod and, moreover, to reduce the transmission of transverse forces to the damper device. The problem here is the lack of precision in the guidance of the damper device on the bearing and thus on the axle. In order to eliminate this problem, the ball joint bearings are provided with a comparatively high preload.

It is the object of the present invention to provide an orthopaedic joint device which impairs the durability of the damper device arranged therein as little as possible and provides improved functionality.

According to the invention, this object is achieved by means of an orthopaedic joint device having the features of the main claim. Advantageous embodiments and developments of the invention are disclosed in the dependent claims, the description and the figures.

The orthopaedic joint device having an upper part and a lower part, which are mounted on one another so as to be pivotable about a joint axis and between which a damper device is arranged in order to provide a resistance to pivoting of the upper part relative to the lower part, and the damper device is mounted on the upper part and the lower part via fastening devices, wherein one fastening device has a head, in which a bearing is arranged, which is supported on an axle, which is mounted in the upper part or the lower part, wherein the axle is mounted rotatably in the upper part or the lower part. The axle can be arranged both on the upper part and on the lower part. In the configuration of an orthopaedic joint device, for example as an artificial knee joint, support is provided via a head, usually on the upper part or on a displaceable component assigned to the upper part, whereas the housing of the damper device is usually arranged on the lower part. In principle, the arrangement and orientation of the head and of the opposite fastening device of the damper device can be interchanged. By means of the bearing in the head, it is ensured that, during the displacement of the upper part relative to the lower part, no or only slight transverse forces and/or torsional moments are introduced into the damper device. The friction usually applied by the bearing, which prevents the upper part from pivoting relative to the lower part beyond the desired damping by the damper device, is eliminated by the rotatable mounting of the axle in the upper part or the lower part. Instead of a rigid axle which is arranged fixedly in the upper part or lower part and at which the mounting requires both a transverse force tolerance and a reduction in the introduction of a torsional moment as well as the enabling of rotation about the longitudinal extent of the axle, it is envisaged according to the invention that the axle itself is mounted so as to be separately rotatable in the upper part or the lower part. This makes it possible to provide very free-running bearings for the axle, which provide precise guidance with low levels of resistance to movement. In this way, it is possible to provide virtually play-free mounting with transverse force decoupling and, at the same time, to avoid the introduction of torsional moments into the damper device.

The bearings of the axle are preferably much more free-running than the bearing in the head, which is supported on the axle, and therefore only, or almost only, the axle rotates in the case of rotation.

Advantageously, the axle is mounted in radial bearings, in particular sliding bearings or needle bearings, in order to achieve high stability and high reliability with a small installation space. Particularly in the case of embodiment of the axle mounting in sliding bearings, it is possible to achieve low-noise and play-free seating of the axle within the respective bearing shells or bearing bushes, Sliding bearings require little maintenance, are robust and have few individual parts, and therefore easy assembly can be achieved with low costs.

The bearing in the head is advantageously designed as a self-aligning ball bearing, self-aligning roller bearing or spherical plain bearing, which enable transverse force decoupling with simultaneous compensation of torsional moments. This prevents torsional moments from being introduced into the damper device. As a result, the linearly displaceable components of the damper device are moved only linearly and are not subjected to any rotation along the longitudinal extent of the cylinder or around the displacement direction. By means of the transverse force decoupling, tilting of the components of the damper device is successfully prevented.

A development of the invention envisages that the bearing is mounted in the head between covering disks. The covering disks serve to shield the bearing from environmental influences and to prevent any lubricant that is present from escaping from the bearing. In addition, the covering disks serve to support and center the bearing with respect to lateral boundaries, in particular the axle mounts for the axle.

The axle can be secured in the upper part or lower part by means of at least one sealing element. Axle mounts, for example axle blocks or bores, into which the axle is pushed or placed and fixed, are arranged or formed in the upper part or lower part. Advantageously, two axle mounts are provided, between which the bearing is arranged in the assembled state on the axle. To secure the axle within the axle mounts, at least one sealing element is advantageously inserted into the respective axle mount. If, for example, there are two axle mounts with through-bores, the axle is secured at the two outer openings by means of at least one sealing element in each case. The sealing element can be designed as a sealing disk, for example, which is screwed or clipped into a recess. The sealing disk can also effect the securing of the axle in the upper part or lower part by means of an additional sealing element, for example a sealing ring or spring ring.

A development of the invention envisages that the axle is mounted so as to be supported axially on the upper part or lower part by means of at least one supporting element. The supporting element can be of elastic design or mounted elastically and has the effect that the axle aligns itself in a self-centering manner within the mounting on the upper part or lower part.

The head, in which the bearing is mounted, is advantageously arranged or formed on a piston rod, the bearing can be mounted on the axle with an interference fit, thus providing a sufficiently high precision of the guidance of both the joint device and the damper device and at the same time ensuring that the rotation about the longitudinal axis of the axle takes place via the separate mounting, in particular sliding contact mounting or mounting in needle bearings, by rotation of the axle.

An exemplary embodiment of the invention is explained in greater detail below with reference to the appended figures. More specifically:

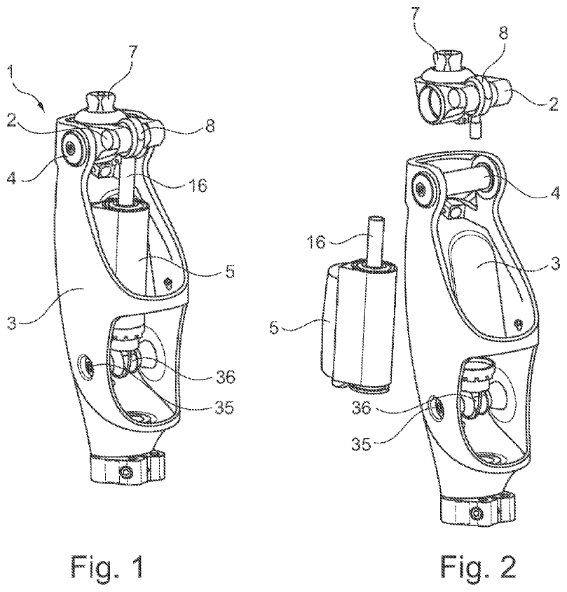

FIG. 1—shows a prosthetic knee joint;

FIG. 2—shows a partially disassembled prosthetic knee joint according to FIG. 1;

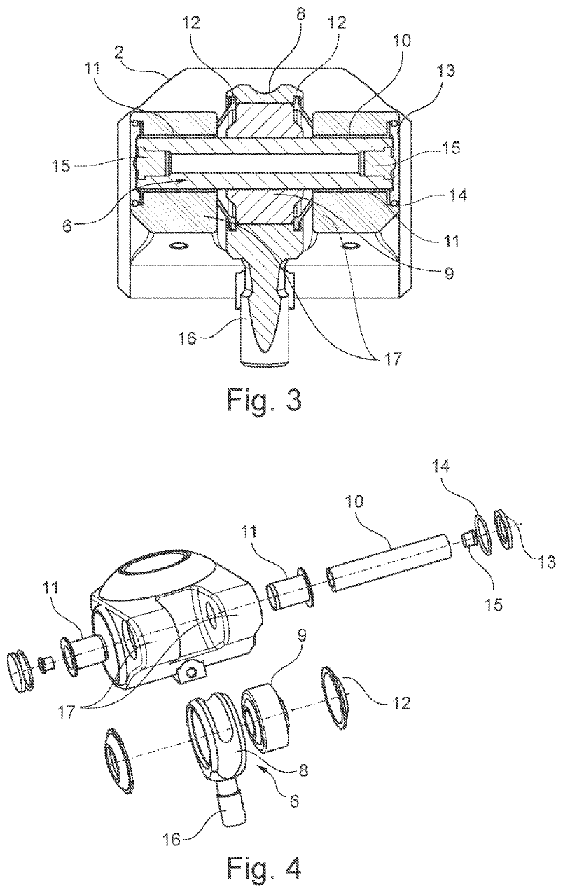

FIG. 3—shows a sectional illustration of part of a joint device; and

FIG. 4—shows an exploded illustration of the components according to FIG. 1.

FIG. 1 shows a perspective illustration of an orthopaedic joint device 1 in the form of a prosthetic knee joint. The orthopaedic joint device 1 has an upper part 2 and a lower part 3, which are mounted on one another so as to be pivotable about a joint axis 4. The lower part 3 is designed as a three-dimensional hollow body, which has an actuator or a damper device 5 with a piston rod 16. At its proximal end, the upper part 2 has a device 7 for fastening a proximal component, e.g. a thigh tube or a thigh socket. In the exemplary embodiment illustrated, the fastening device 7 is designed as a pyramid adapter; other configurations are likewise possible. Furthermore, a head 8, which is arranged or fastened on a proximal end of the piston rod 16, is mounted on the upper part 2 in such a way that it can be pivoted about an axle 10, which will be explained in greater detail below. Furthermore, the distal end of the damper device 5 is mounted so as to be pivotable about an axis 35 at a distal bearing location 36. The damper device 5 can be secured in a releasable manner both at the distal bearing location 36 and at the head 8, in particular by means of a screw connection or snap connection.

FIG. 2 shows the orthopaedic joint device according to FIG. 1 in a partially assembled illustration. The head 8 is separated from the piston rod 16, and the distal end of the damper device 5 is likewise detached from the distal bearing location 36. The joint axis 4, which extends transversely through the lower part 3, is designed as a pin and, in the assembled state, extends through a through-hole within the upper part 2. Distal prosthesis components e.g. a lower leg tube or a prosthetic foot, can be arranged at the distal end of the lower part 3. The distal bearing location 36 permits, in particular, pivoting of the damper device 5 exclusively about the axis 35, which is oriented substantially parallel to the joint axis 4. The head 8 is mounted so as to be pivotable about its axis, which will be explained in greater detail below.

The damper device 5, as illustrated in FIGS. 1 and 2, can be designed as a hydraulic damper device, and a pneumatic damper device is also envisaged as an alternative. It is also possible to use damper devices which operate on the basis of magnetorheological effects. As an alternative to a passive configuration, the damper device 5 can also be designed as an actuator, e.g. as an electric drive, which, by means of appropriate circuitry, enables and permits displacement of the upper part 2 relative to the lower part 3 and opposes this displacement with a resistance.

In addition to a configuration of the orthopaedic joint device 1 as a prosthetic knee joint, it can also be designed as an orthotic knee joint or some other joint device.

FIG. 3 shows a sectional view of part of an orthopaedic joint device 1, namely an upper part 2, which is mounted on the lower part 3 (not illustrated) so as to be pivotable about the joint axis 4 (likewise not illustrated). The damper device 5 with the piston rod 16 and the head 8 is mounted on the upper part 2, which can be designed, for example, as a knee head with devices for fastening a thigh socket. The piston rod 16 is coupled to a piston, which is mounted in a longitudinally displaceable manner on a cylinder and is part of a hydraulic damper. Arranged at the upper end of the piston rod 16, within the head 8, which has a bore, is a bearing 9, which is, in particular, an interference fit. The bearing 9 is designed as a self-aligning ball bearing, self-aligning roller bearing or spherical plain bearing and is supported on an axle 10, which is supported in two axle mounts 17. The axle mounts 17 are arranged on both sides of the head 8. Arranged between the axle mounts 17 and the bearing 9 are two covering disks 12 which, on the one hand, provide sealing of the bearing 9 against external influences and, on the other hand, effect centering of the head 8 between the axle mounts 17. The covering disks 12 are of elastic and dish-like design and are inserted at their outer circumference into grooves within the head 8. The bearing 9 is fixed on the axle 10, likewise by means of an interference fit. Together with the bearing 9, the head 8 on the piston rod 16 forms the fastening device 6 by means of which the damper device 5 is fixed directly on the upper part 2 via the axle 10.

The axle 10 itself is oriented orthogonally to the longitudinal extent of the piston rod 16 and is mounted rotatably on the upper part 2, within the axle mounts 17, by means of two sliding bearings 11. The sliding bearings 11 are designed as sliding bearing bushes and are an interference fit in the axle mounts 17. A cover 13 with a circumferential O-ring seal 14 is provided as a means of preventing them from falling axially out of the axle mounts 17. The cover 13 and the O-ring seal 14 serve as sealing elements which, on the one hand, prevent the penetration of dust and moisture into the sliding bearing pairing between the axle 10 and the sliding bearing bush 11. At the same time, they secure both the sliding bearing bush 11 and the axle mechanically. By means of the cover 13 and the O-ring seal 14, which is inserted on the outside in a circumferential groove within the axle mount 17, positive locking of the cover 13 is provided. This prevents the axle 10 from being displaced axially along its longitudinal extent. For self-centering, two supporting elements 15 are arranged within the axle 10, said supporting elements being designed as elastomer elements and being supported on the inside on the covers 13. The supporting elements 15 enable the axles to be displaced axially to a small extent, thereby preventing the transmission of transverse forces from the axle mount 17 to the piston rod 10.

If the upper part 2 is displaced about the joint axis 4, axle 10 moves on a circular path about the joint axis 4 and correspondingly moves the bearing 9 and thus also the head 8 on a circular path. During this process, the piston rod 16 is moved both downward in the direction of the distal bearing location 36 and out of the plane of the drawing. The rotary movement which occurs during this process between the upper part 2 and the head 8 is made possible by the rotatable mounting of the axle 10 in the sliding bearing bushes 11. If alignment errors occur between the axle 10 and the joint axis 4 or if deformations due to external forces or wear are present, transverse forces and torsional moments are absorbed and compensated by the bearing 9. This prevents tilting and twisting of the piston rod 16, ensuring that the piston fastened to the piston rod 16 is always guided linearly and coaxially with respect to the longitudinal extent of the cylinder. The sliding bearings 11 can absorb high static forces and permit play-free, low-noise rotation of the axle 10 relative to the upper part 2. As a result, pivoting about the joint axis 4 is unaffected, and therefore resistance to pivoting is modified exclusively by the settings within the damper device 5. Smooth pivoting without the introduction of transverse forces and with minimal play allows long-term, trouble-free and silent operation of the orthopaedic joint device 1.

FIG. 4 shows an exploded illustration of the components according to FIG. 3. The piston rod 16 with the integrally formed head as part of the damper device 5 serves to receive the bearing 9, which is designed as a self-aligning ball bearing, self-aligning roller bearing or spherical plain bearing. The two covering disks 12, which are likewise inserted in positive engagement within the head 8, are arranged on both sides of the bearing 9. Owing to the dish-shaped, resilient configuration, the covering disks 12 center the head 8 centrally within the axle mounts 17.

Inserted within the axle mounts 17 are the two sliding bearing bushes 11; in which the axle 10, which passes through the bearing 9, is rotatably mounted. The two elastic supporting elements 15 or elastically mounted supporting elements 15 are arranged within the hollow-drilled axle 10 and are supported on the two sealing disks 13, which are fixed positively by means of O-rings 14 within the axle mounts 17.

1. An orthopaedic joint device having an upper part (2) and a lower part (3), which are mounted on one another so as to be pivotable about a joint axis (4) and between which a damper device (5) is arranged in order to provide a resistance to pivoting of the upper part (2) relative to the lower part (3), and the damper device (5) is mounted on the upper part (2) and the lower part (3) via fastening devices (6, 7), wherein one fastening device (6) has a head (8), in which a bearing (9) is arranged, which is supported on an axle (10), which is mounted in the upper part (2) or lower part (3), characterized in that the axle (10) is mounted rotatably in the upper part (2) or lower part (3).

2. The orthopaedic joint device as claimed in claim 1, characterized in that the axle (10) is mounted in radial bearings, in particular sliding bearings (11) or needle bearings.

3. The orthopaedic joint device as claimed in claim 1, characterized in that the bearing (9) in the head (8) is designed as a self-aligning ball bearing, a self-aligning roller bearing or a spherical plain bearing.

4. The orthopaedic joint device as claimed in claim 1, characterized in that the bearing (9) is mounted between covering disks (12) in the head (8).

5. The orthopaedic joint device as claimed in claim 1, characterized in that the axle (10) is secured in the upper part (2) or lower part (3) by means of at least one sealing element (13, 14).

6. The orthopaedic joint device as claimed in claim 1, characterized in that the axle (10) is mounted so as to be supported axially on the upper part (2) or lower part (3) by means of at least one supporting element (15).

7. The orthopaedic joint device as claimed in claim 6, characterized in that the supporting element (15) is of elastic design or is elastically mounted.

8. The orthopaedic joint device as claimed in claim 1, characterized in that the head (8) is arranged or formed on a piston rod (16).

9. The orthopaedic joint device as claimed in claim 1, characterized in that the bearing (9) is mounted on the axle (10) with an interference fit.
